(19) United States
(12) Patent Application Publication
(10) Pub. No.: US 2016/0193131 A1
(43) Pub. Date: Jul. 7, 2016

(54) METHOD FOR REDUCING SURFACE FREE ENERGY AND COMPOSITION HAVING REDUCED SURFACE FREE ENERGY

(71) Applicants: KANEKA CORPORATION, Osaka-shi, Osaka (JP); NATIONAL INSTITUTE OF ADVANCED INDUSTRIAL SCIENCE AND TECHNOLOGY, Tokyo (JP)

(72) Inventors: Satohiro YANAGISAWA, Tokyo (JP); Masashi IZUMIDA, Takasago-shi (JP); Toshiaki TAIRA, Tsukuba-shi (JP); Tomohiro IMURA, Tsukuba-shi (JP); Dai KITAMOTO, Tsukuba-shi (JP)

(73) Assignees: KANEKA CORPORATION, Osaka-shi, Osaka (JP); NATIONAL INSTITUTE OF ADVANCED INDUSTRIAL SCIENCE AND TECHNOLOGY, Tokyo (JP)

(21) Appl. No.: 14/911,636

(22) PCT Filed: Aug. 11, 2014
(86) PCT No.: PCT/JP2014/071224

§ 371 Date: Feb. 11, 2016

(30) Foreign Application Priority Data
Aug. 12, 2013 (JP) 2013-167766
May 1, 2014 (JP) 2014-094643

(51) Int. Cl. A61K 8/64; A61K 8/02; A01N 31/02; A61Q 17/00; A61Q 19/10; A61K 8/34; A01N 43/72; A61Q 1/14

(52) U.S. Cl. CPC A61K 8/64; A01N 43/72; A61K 8/0208; A61Q 1/14; A61Q 17/005; A61Q 19/10; A61K 8/34; A01N 31/02; A61K 2800/75; A61K 2800/596

(57) ABSTRACT

An objective of the present invention is to provide a method for reducing surface free energy of an organic solvent and of a mixed solution of water and a water-miscible organic solvent, a composition which has a reduced surface free energy of an organic solvent and of a mixed solution of water and a water-miscible organic solvent and which has high environmental suitability and safety to a living body, and a wet wiper and an antiseptic solution which contains the composition The method for reducing surface free energy of an organic solvent or a mixed solvent of water and a water-miscible solvent according to the present invention is characterized in comprising the step of adding surfactin or a salt thereof to the organic solvent or mixed solvent.

### TECHNICAL FIELD

The present invention relates to a method for reducing surface free energy of an organic solvent and the like, a composition which contains an organic solvent but of which surface free energy is reduced, and a wet wiper and an antiseptic solution which contain the composition.

### BACKGROUND ART

A surfactant forms a micelle, a vesicle, a lamella structure or the like in a solvent if a concentration is sufficiently high and exhibits effects such as the reduction of surface free energy, since a surfactant has a hydrophilic group and a hydrophobic group in the structure thereof. Thus, a surfactant is used, for example, for homogeneously mixing a polar material and a non-polar material. Specifically, a surfactant is used as a detergent component to remove a non-polar stain by separating the stain into a detergent liquid, or in foods to allow a non-polar material such as a flavor to be homogeneously dispersed in an aqueous solvent. In addition, for example, a surfactant has also a function to allow a detergent to enter a narrow space, since surface tension of a solvent is reduced due to the reduction of surface free energy.

However, a surfactant is mainly used in an aqueous solvent, and the above-described effects are not obtained or almost not obtained when a surfactant is used in an organic solvent. Even when a surfactant is used in an aqueous solvent, if a water-miscible organic solvent is contained in the aqueous solvent, there is a problem that the above effects may be significantly decreased.

On the one hand, a surfactant having a fluorinated alkyl chain is known to exhibit surface activity even in an organic solvent (Patent Document 1). However, in general, a so-called fluorine-containing compound can never be mixed in a wet wiper which may be used for removing cosmetics or for wiping the buttock of a baby, or in an antiseptic solution for the purpose of disinfection of hands and fingers, since such a compound has low environmental suitability and low biocompatibility.

A wet wiper having reduced skin irritancy and good wiping properties is described in Patent Document 2. In addition, Patent Document 3 discloses a cosmetic formulation which supports the antimicrobial action of an alcohol in a synergistic manner and of which skin irritancy is low.

### PRIOR ART DOCUMENT

### Patent Document

Patent Document 1: JP 5142267 B

Patent Document 2: JP 2012-153736 A

Patent Document 3: JP 2012-527411 T

### DISCLOSURE OF THE INVENTION

### Problems to be Solved by the Invention

As described above, various compositions which are applicable to a wet wiper have been developed in view of skin irritancy and the like.

However, the major component of the wet wiper described in Patent Document 2 is a salt of condensate of N-lauroyl-L-glutamic acid and L-lysine, or the like, and the major component of the composition described in Patent Document 3 is sorbitan monocaprylate. Even though the components may exhibit surface activity under a normal condition, it is considered that the components may not exhibit sufficient surface activity in the presence of an organic solvent similarly to a conventional surfactant.

Under such circumstances, an objective of the present invention is to provide a method for reducing surface free energy of an organic solvent and of a mixed solution of water and a water-miscible organic solvent, a composition which has a reduced surface free energy of an organic solvent and of a mixed solution of water and a water-miscible organic solvent and which has high environmental suitability and safety to a living body, and a wet wiper and an antiseptic solution which contains the composition.

### Means for Solving the Problems

The inventors of the present invention made extensive studies to solve the above problems. As a result, the inventors completed the present invention by finding that surfactin, which is a natural surfactant, can effectively reduce the surface free energy of an organic solvent and has high environmental suitability and high biocompatibility.

Hereinafter, the present invention is described.

[1] A method for reducing surface free energy of an organic solvent or a mixed solvent of water and a water-miscible solvent, comprising the step of adding surfactin represented by the following formula (I) or a salt thereof to the organic solvent or mixed solvent,

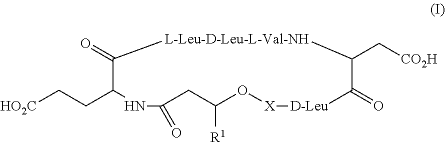

wherein ‘X’ is a residue of an amino acid selected from leucine, isoleucine and valine; and R¹ is a C₉₋₁₈ alkyl group.

[2] The method according to the above [1], wherein the surfactin or salt thereof is added in an amount of not less than 0.01 mass % to the organic solvent or mixed solvent. The critical micelle concentration of the surfactin (I) in 100% of water is about 0.0003 to 0.003 mass %; therefore, when the ratio is 0.01 mass % or more, a micelle, a vesicle or a lamella structure is formed more reliably and an interfacial activity in an organic solvent and the like is exerted more surely.

[3] The method according to the above [1] or [2], wherein a ratio of the water-miscible solvent in the mixed solvent is adjusted to not less than 5 vol %. A conventional surfactant cannot sufficiently exert an interfacial activation effect even in the presence of a mixed solvent containing water in the presence of an organic solvent; therefore, the above requirement has significance to clarify differences between a prior art and the present invention.

[4] A composition, comprising surfactin represented by the following formula (I) or a salt thereof and an organic solvent or a mixed solvent of water and a water-miscible solvent,

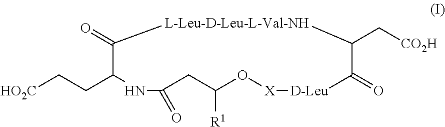

wherein ‘X’ is a residue of an amino acid selected from leucine, isoleucine and valine; and R¹ is a C₉₋₁₈ alkyl group.

[5] The composition according to the above [4], wherein the surfactin or salt thereof is contained in an amount of not less than 0.01 mass % to the organic solvent or mixed solvent. Due to the above-described reason, when the ratio is 0.01 mass % or more, a micelle, a vesicle or a lamella structure is formed more reliably and an interfacial activity in an organic solvent and the like is exerted more surely.

[6] The composition according to the above [4] or [5], wherein a ratio of the water-miscible solvent in the mixed solvent is not less than 5 vol %. The requirement has significance to clarify differences between a prior art and the present invention as the above.

[7] A wet wiper, comprising the composition according to any one of the above [4] to [6].

[8] An antiseptic solution, comprising the composition according to any one of above [4] to [6].

### Effect of the Invention

The surfactin (I) according to the present invention is easily decomposed after use, since the surfactin (I) is a peptide compound. The surfactin (I) therefore has high environmental suitability as well as high biocompatibility and is safe. In addition, unlike a conventional surfactant, the surfactin (I) according to the present invention enables the surface free energy of an organic solvent itself or a mixed solution containing an organic solvent to be reduced. When the surfactin (I) according to the present invention has a concentration equal to or higher than the critical micelle concentration, a micelle, vesicle, lamellar structure and the like are formed, and it becomes possible to homogeneously mix a polar material and a non-polar material. The present invention is therefore extremely useful for industries, since the properties of a formulation which contains an organic solvent can be improved by the present invention.

### BRIEF DESCRIPTION OF THE DRAWINGS

FIG. 1 is a graph showing a particle size distribution of micelles which are formed from the surfactin according to the present invention in methanol.

FIG. 2 is a graph showing a particle size distribution of micelles which are formed from the surfactin according to the present invention in ethanol.

FIG. 3 is a polarizing microscope photograph of lamellar liquid crystal which is formed from the surfactin according to the present invention in ethanol.

FIG. 4 is a graph showing a particle size distribution of micelles which are formed from the surfactin according to the present invention in 20 vol % ethanol aqueous solution.

FIG. 5 is a graph showing measurement results of surface tension-reducing ability of sodium dodecyl sulfate and the surfactin according to the present invention for 20 vol % ethanol aqueous solution.

FIG. 6 is a graph showing the result of analyzing the formation of the secondary structure of the surfactin according to the present invention in an acetone solution by a circular dichroism dispersion meter.

FIG. 7 is a graph showing measurement results of surface tension-reducing ability of sodium dodecyl sulfate and the surfactin according to the present invention for an acetone aqueous solution.

FIG. 8 is a graph showing the result of analyzing the formation of the secondary structure of the surfactin according to the present invention in a methanol solution by a circular dichroism dispersion meter.

FIG. 9 is a graph showing the result of analyzing the formation of the secondary structure of the surfactin according to the present invention in an ethanol solution by a circular dichroism dispersion meter.

FIG. 10 is a graph showing the result of analyzing the formation of the secondary structure of the surfactin according to the present invention in a tetrahydrofuran solution by a circular dichroism dispersion meter.

### MODE FOR CARRYING OUT THE INVENTION

The method for reducing surface free energy of an organic solvent or a mixed solvent of water and a water-miscible solvent according to the present invention is characterized in comprising the step of adding the surfactin (I) or salt thereof to the organic solvent or mixed solvent.

In the present invention, the term “organic solvent” means an organic compound which is a liquid at ordinary temperature and ordinary pressure and which is not water regardless of whether the organic solvent is water-miscible or not. The organic solvent is exemplified by an alcohol solvent such as methanol, ethanol and isopropanol; a polyol solvent such as ethylene glycol, propylene glycol, diethylene glycol and dipropylene glycol; an ether solvent such as diethyl ether and tetrahydrofuran; a ketone solvent such as acetone; a nitrile solvent such as acetonitrile; an amide solvent such as dimethylformamide and dimethylacetamide; a sulfoxide solvent such as dimethylsulfoxide; a carboxylic acid solvent such as formic acid and acetic acid; an ester solvent such as ethyl acetate; an aliphatic hydrocarbon solvent such as hexane; an aromatic hydrocarbon solvent such as benzene, toluene and xylene; a halogenated aliphatic hydrocarbon solvent such as dichloromethane and chloroform; and a halogenated aromatic hydrocarbon solvent such as chlorobenzene.

The term “water-miscible solvent” means an organic solvent, for example, not less than 5 g of which can be homogeneously mixed in 100 mL of water at 20° C. in the absence of a solute or in the presence of the surfactin (I) or salt thereof. The water-miscible solvent is exemplified by an alcohol solvent, a polyol solvent, an ether solvent, a ketone solvent, a nitrile solvent, an amide solvent, a sulfoxide solvent and a carboxylic acid solvent among the above-described organic solvent. A water-miscible solvent which can be mixed with water with no limit is preferred.

When a mixed solvent of water and a water-miscible solvent is used, the mixing ratio thereof is not particularly restricted but the ratio of a water-miscible solvent in the mixed solvent is preferably not less than 5 vol %. The ratio is more preferably not less than 10 vol %, and even more preferably not less than 20 vol %, not less than 40 vol %, not less than 50 vol %, not less than 60 vol % or not less than 80 vol %. The upper limit of the ratio is not particularly restricted and may be extremely close to 100 vol %. However, the ratio is preferably not more than 99 vol %, more preferably not more than 98 vol %, even more preferably not more than 96 vol %, and particularly preferably not more than 95 vol %, since the surface active effect of the surfactin (I) may be more effectively exerted when the water ratio is large.

In the present invention, the surface free energy of the above-described organic solvent or mixed solvent is reduced by adding the surfactin (I) or salt thereof to the organic solvent or mixed solvent. The surfactin (I) has a small environmental load and safety to a human body, since the surfactin (I) is a peptide compound.

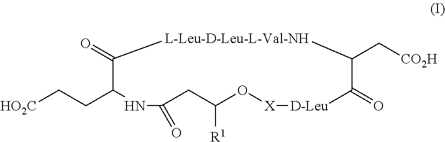

wherein ‘X’ is a residue of an amino acid selected from leucine, isoleucine and valine; R¹ is a C₉₋₁₈ alkyl group.

Although the amino acid residue as ‘X’ may be either in a L-form or a D-form, the L-form is preferred.

The term “C₉₋₁₈ alkyl group” means a linear or branched monovalent saturated hydrocarbon group having not less than 9 and not more than 18 carbon atoms. The example thereof includes n-nonyl, 6-methyloctyl, 7-methyloctyl, n-decyl, 8-methylnonyl, n-undecyl, 9-methyldecyl, n-dodecyl, 10-methylundecyl, n-tridecyl, 11-methyldodecyl, n-tetradecyl, n-pentadecyl, n-hexadecyl, n-heptadecyl and n-octadecyl.

Either one of the surfactin (I) may be used or not less than two of the surfactin (I) may be used. For example, two or more surfactin (I) of which C₉₋₁₈ alkyl groups are different may be used.

The surfactin salt (I) can be isolated from a culture broth prepared by culturing a microorganism such as a strain belonging to Bacillus subtilis in accordance with a known method. The surfactin (I) may be a purified product or an unpurified product. For example, a culture broth may be directly used as the unpurified product. Alternatively, the product of the surfactin (I) obtained by a chemical synthesis method may be similarly used.

The counter cation which constitutes the salt of the surfactin (I) is not particularly restricted and exemplified by an alkali metal ion and an ammonium ion.

The alkali metal ion for the salt of the surfactin (I) is not particularly restricted and exemplified by a lithium ion, a sodium ion, a potassium ion or the like. When two or more alkali metal ions are used, the ions may be the same or different from each other.

The example of a substituent of the ammonium ion includes an organic group, for example, a C₁₋₄ alkyl group such as methyl, ethyl, n-propyl, isopropyl, n-butyl and t-butyl; a C₇₋₁₄ aralkyl group such as benzyl, methylbenzyl and phenylethyl; and a C₆₋₁₂ aryl group such as phenyl, toluyl and xylyl. The ammonium ion is exemplified by a tetramethylammonium ion, a tetraethylammonium ion and a pyridinium ion.

In the salt of the surfactin (I), two counter cations may be the same or different from each other. In addition, one of the carboxy groups may be in the state of —COOH or —COO⁻.

A means and condition for adding the surfactin (I) or salt thereof is not particularly restricted and appropriately selected. For example, the surfactin (I) or salt thereof is added to an organic solvent or a mixed solvent of water and a water-miscible solvent, and then the mixture is stirred. The temperature at the time may be an ordinary temperature, and may be specifically not less than about 10° C. and not more than about 50° C. The stirring time is not particularly restricted, and for example, may be not less than about 30 seconds and not more than about 1 hour.

A ratio of the surfactin (I) or salt thereof to an organic solvent or a mixed solvent of water and a water-miscible solvent is not restricted, and for example, may be appropriately adjusted depending on the purpose, the kind and amount of an additive component, or the like. The purpose is exemplified by reduction of surface tension, and dissolution and homogeneous dispersion of an additive component. For example, the above-described ratio to be added is preferably not less than 0.01 mass %. When the ratio is not less than 0.01 mass %, a micelle, vesicle or lamellar structure is formed even in the presence of an organic solvent more surely and surfactant activity in an organic solvent and the like is obtained more certainly. In addition, the surfactin or salt thereof forms a stable secondary structure and further forms an aggregate such as a micelle in an organic solvent and the like; as a result, surface free energy of an organic solvent and the like is reduced. The ratio is more preferably not less than 0.05 mass %, even more preferably not less than 0.1 mass %, even more preferably not less than 0.2 mass %, and even more preferably not less than 0.5 mass %. On the one hand, the upper limit is not particularly restricted; however, the ratio is preferably not more than 20 mass %, more preferably not more than 10 mass %, even more preferably not more than 5 mass %, and particularly preferably not more than 2 mass %.

The composition of the present invention contains an organic solvent; however, the surfactin (I) forms a micelle, vesicle, lamellar structure and the like in the composition, and the surface tension thereof is reduced. As a result, a component which is hardly dissolved or homogeneously dispersed by a general surfactant can be dissolved or homogeneously dispersed in the composition, and the composition can enter narrow space into which a general composition cannot enter. In addition, though foaming property is generally lost in the presence of an organic solvent, foaming property can be maintained in a certain degree by using the present invention composition of which function can be exhibited even in the presence of an organic solvent.

The composition of the present invention having the above-described properties can be applied to a product which contains an organic solvent. A product containing an organic solvent has a problem that a function of a surfactant cannot be sufficiently exhibited due to the organic solvent. On the one hand, when the surfactin according to the present invention is mixed, a component which cannot be blended due to non-polarity can be dissolved or homogeneously dispersed, the amount of such a component to be mixed can be increased, it becomes possible to send such a component to a narrow space to which the component has never reached without the surfactin (I) in order to exhibit the cleaning effect thereof, and foaming property may be maintained in a certain degree even in the presence of an organic solvent.

A product which contains an organic solvent is not particularly restricted, and exemplified by a wet wiper such as a wet tissue which is used for removing cosmetics or cleaning bottom of an infant; a medical or household antiseptic solution for antisepticizing fingers or the like; a cosmetic product and toiletry product, such as cream, gel, lotion, shampoo, a product for a shower bath, deodorant, antiperspirant, sunscreen product, cosmetic product for ornament, liquid tooth dentifrice and mouth wash solution; a fiber product; rubber/plastic product; product for civil engineering and construction; paper/pulp product; machine/metal product; cleaning product; beverage and food; paint/ink product; environmental preservation product; agricultural/fertilizer product; information industry product; other industrial detergent which contain an organic solvent.

The composition and product according to the present invention may contain other additive component depending on the use application thereof. Such other additive component is exemplified by a polysaccharide thickener such as guar gum and xanthane gum; a cellulose compound such as hydroxypropylcellulose and carboxymethylcellulose; a carboxyvinyl polymer such as an acrylic acid polymer and an acrylic acid copolymer; a silicone compound; a colorant; a pH adjuster; a plant extract; a preservative; a chelating agent; a vitamin preparation; a medicinal ingredient such as an anti-inflammatory drug; a fragrance; a ultraviolet absorber; and an antioxidant. A conventional surfactant may be mixed in the composition and product according to the present invention in addition to the surfactin (I); however, it is preferred that all of the surfactant in the composition and product is the surfactin (I).

The present application claims the benefit of the priority dates of Japanese patent application No. 2013-167766 filed on Aug. 12, 2013, and of Japanese patent application No. 2014-94643 filed on May 1, 2014. All of the contents of the Japanese patent application No. 2013-167766 filed on Aug. 12, 2013, and Japanese patent application No. 2014-94643 filed on May 1, 2014, are incorporated by reference herein.

### EXAMPLES

Hereinafter, the present invention is described in more detail with Examples. However, the present invention is not restricted to the following Examples in any way, and it is possible to work the present invention according to the Examples with an additional appropriate change within the range of the above descriptions and the following descriptions. Such a changed embodiment is also included in the technical scope of the present invention.

### Example 1

### Confirmation of Micelle Formation in Organic Solvent

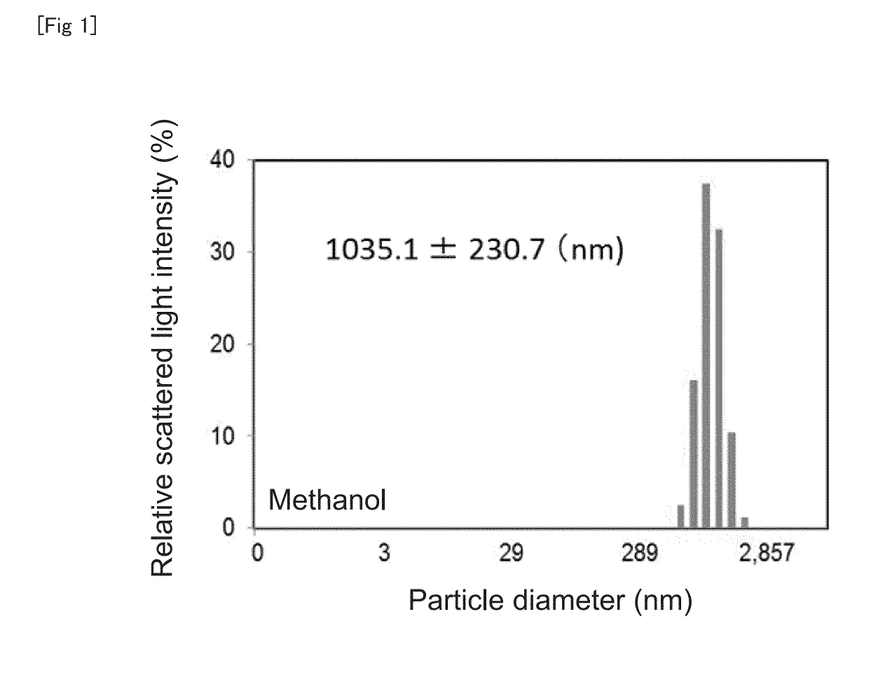

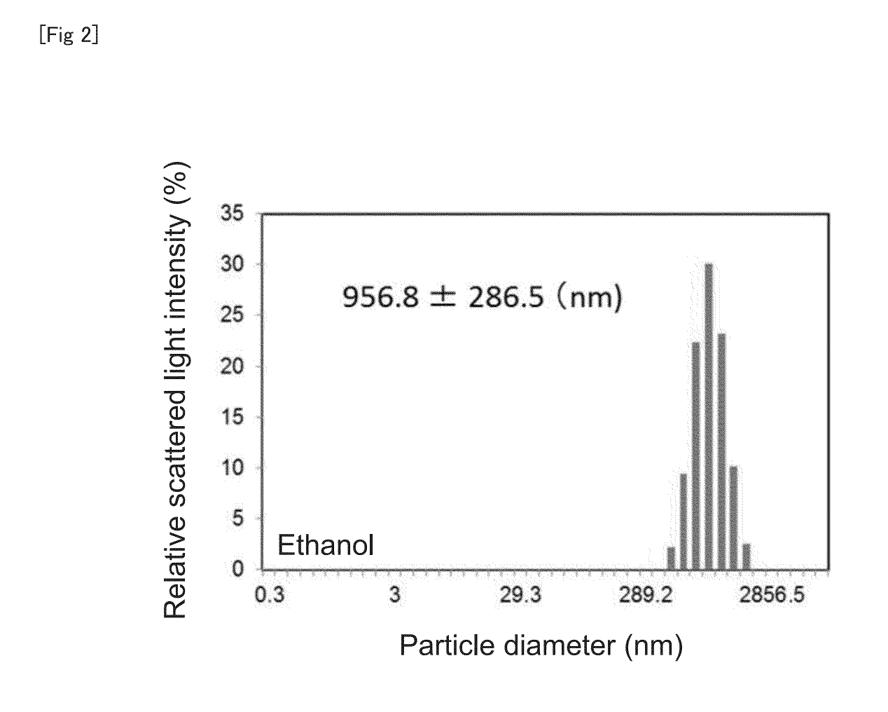

In a test tube, surfactin sodium salt (hereinafter, referred to as “SFNa”) and methanol (5 mL) or ethanol (5 mL) were added so that SFNa concentration became 2 mass %. The mixture was stirred using a vortex mixer for 3 minutes. A particle size distribution of the particles which were contained in each obtained dispersion was measured using a dynamic light scattering measuring device (product name: DLS-7000, manufactured by OTSUKA ELECTRON Co., Ltd.). In the measurement, Ar laser (λ=488 nm) was used as a light source and a scattering angle was adjusted to 90°. The result of the methanol dispersion is shown by FIG. 1 and the result of the ethanol dispersion is shown by FIG. 2.

As FIGS. 1 and 2, it was found that SFNa forms a large micelle having each average particle diameter of 1035.1±230.7 nm and 956.8±286.5 nm in the both cases of methanol (FIG. 1) and ethanol (FIG. 2). When SFNa was added and mixed in an alcohol, the mixture became transparent by naked eyes. It was however revealed that surfactin exhibits self-assembling capability in an alcohol and can form a large aggregate as described above.

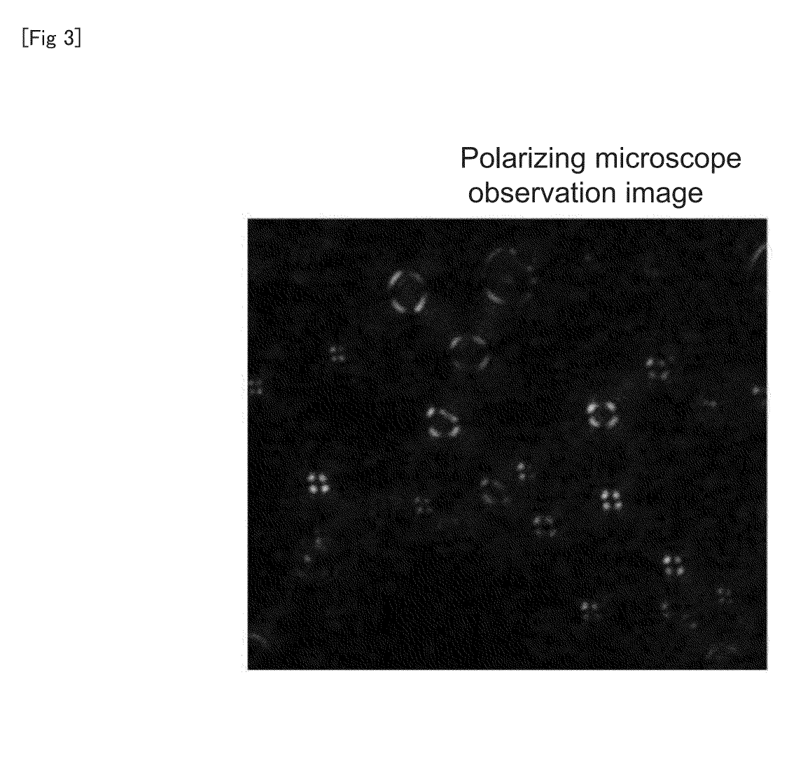

In addition, the above each dispersion was left to stand for one day at 25° C. As a result, the methanol dispersion remained transparent by naked eyes; on the one hand, there were deposits in the ethanol dispersion. The depositions were observed using a polarization microscope (“ECLIPSE E600”, manufactured by NIKON CORPORATION). The result is shown as FIG. 3. As FIG. 3, a Maltese Cross image which is characteristic of lamellar liquid crystal was observed. It was therefore found that SFNa can form not only the above-described huge micelle but also a lamellar liquid crystal in ethanol. A lamellar liquid crystal may incorporate both of a hydrophilic substance and a lipophilic substance inside, since a lamellar liquid crystal has both of a hydrophilic part and a lipophilic part.

### Example 2

### Confirmation of Effect of Reducing Surface Free Energy in Organic Solvent

Then, an experiment for confirmation of effect of reducing surface free energy by SFNa in organic solvent was carried out. Specifically, SFNa and diethylene glycol (10 mL) was added in a test tube so that SFNa concentration became 2 mass % similarly to the above-described Example 1. The mixture was stirred using a vortex mixer for 3 minutes. The dispersion was transferred to a petri dish and left to stand. The surface tension was measured at 25° C. using a high function surface tension measuring device (“DY-500”, manufactured by Kyowa Interface Science Co., Ltd.). In addition, the surface tensions of diethylene glycol only and 2 mass % diethylene glycol solution of sodium dodecyl sulfate, i.e. “SDS”, were similarly measured as control. The result is shown in Table 1.

TABLE 1

| | Diethylene glycol only | 2% SDS solution | 2% SFNa dispersion |
|---|---|---|---|
| Surface | 45.2 | 44.5 | 39.6 |
| tension | | | |
| (mN/m) | | | |

As the above-described result, in the case of sodium dodecyl sulfate, i.e. “SDS”, which is a general surfactant, the surface tension of diethylene glycol could not be reduced. On the one hand, when SFNa was used, the surface tension could be clearly reduced. It was thus demonstrated that surface free energy can be reduced even in an organic solvent by SFNa. It is contemplated as the result in the above-described Example 1 that SFNa forms an aggregate such as micelle in a solvent; as a result, surface free energy of a solvent can be reduced by SFNa according to the present invention.

### Example 3

### Confirmation of Micelle Formation and Effect of Reducing Surface Free Energy in Mixed Solvent

An experiment for confirmation of effect of reducing surface free energy by SFNa in a mixed solvent of water and a water-miscible solvent was also carried out. Specifically, SFNa and 20 vol % ethanol aqueous solution (10 mL) was added in a test tube so that SFNa concentration became 2 mass % similarly to the above-described Example 1. The mixture was stirred using a vortex mixer for 3 minutes. In addition, 2 mass % SDS solution was obtained as control by dissolving SDS in 20 vol % ethanol aqueous solution.

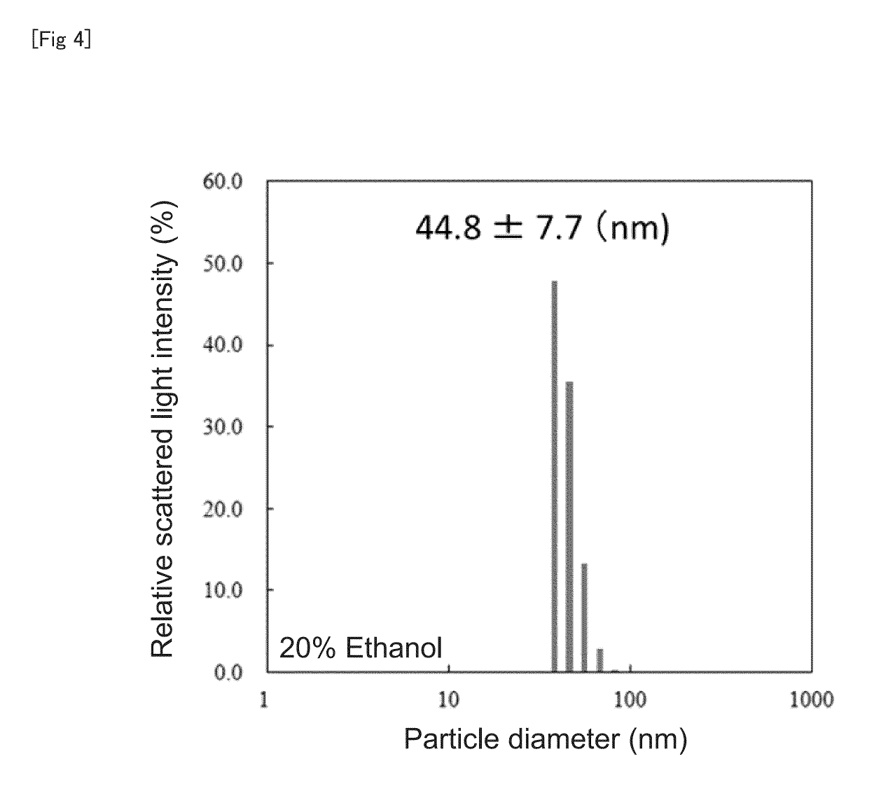

First, an association behavior of SFNa was evaluated by a dynamic light scattering method as the above-described Example 1. The result is shown in FIG. 4. As FIG. 4, a micelle having an average particle diameter of 44.8±7.7 nm was observed. On the one hand, a micelle cannot be observed in the case of SDS even in the same concentration.

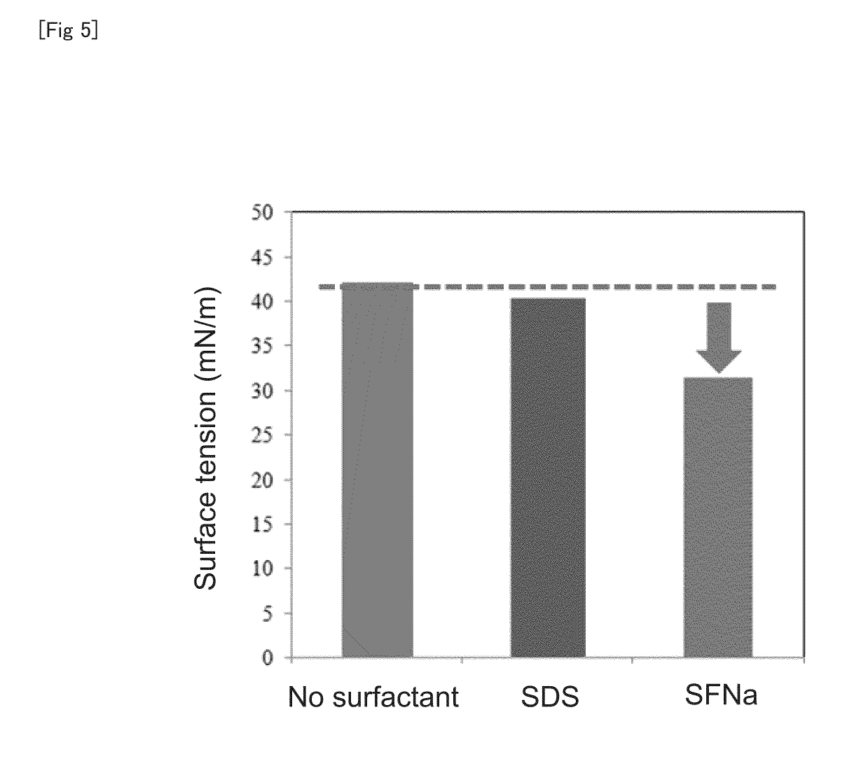

In addition, the surface tension reducing ability of SFNa and SDS in 20 vol % ethanol aqueous solution was evaluated at 25° C. similarly to the above-described Example 2. The result is shown in FIG. 5. As FIG. 5, SDS could not reduce the surface tension of 20 vol % ethanol aqueous solution; on the one hand, SFNa could clearly reduce the surface tension. It was thus demonstrated that SFNa forms a micelle and can reduce surface free energy even in a mixed solvent water and a water-miscible solvent.

### Example 4

### Confirmation of Effect of Reducing Surface Free Energy in Organic Solvent

Furthermore, surface tension was measured similarly to the above-described Example 2 except that the organic solvent was changed from diethylene glycol to dimethylsulfoxide, i.e. DMSO. The result is shown in Table 2.

TABLE 2

| | DMSO only | 2% SDS solution | 2% SFNa dispersion |
|---|---|---|---|
| Surface | 43.5 | 43.4 | 39.2 |
| tension | | | |
| (mN/m) | | | |

As the result shown in Table 2, SDS could not reduce the surface tension of DMSO. On the one hand, when SFNa was used, the surface tension could be clearly reduced. It was thus demonstrated that surface free energy can be reduced even in an organic solvent by SFNa.

### Example 5

### Confirmation of Secondary Structure Formation in Organic Solvent

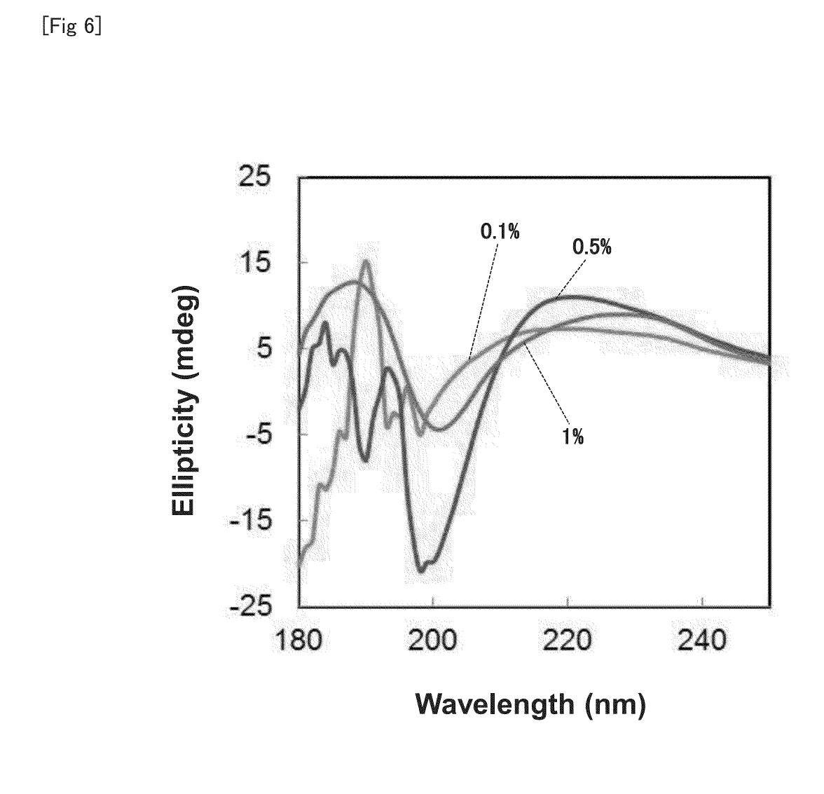

Similarly to the above-described Example 1, SFNa and acetone (5 mL) was added in a test tube. The mixture was stirred using a vortex mixer for 3 minutes in order to prepare 0.1 mass %, 0.5 mass % or 1 mass % SFNa solution in acetone. Then, secondary structure formation of SFNa in the prepared acetone solutions was measured using a circular dichroism dispersion meter (“J-820”, manufactured by JASCO Corporation). The result is shown in FIG. 6.

It was demonstrated in FIG. 6 that a negative peak was observed near 200 nm. It was clearly demonstrated from the result that while it has been known that SFNa forms secondary structure in water, SFNa forms secondary structure in not only water but also an acetone solution. In addition, it was confirmed that when the concentration of SFNa was higher, secondary structure formation was facilitated, since larger peak could be observed in such a case. Considering the results of Example 5 in addition to Examples 1 to 4, it is contemplated that surface free energy of an organic solvent can be reduced by the aggregate such as micelle of a stable secondary structure formed by SFNa in not only water but also an organic solvent.

### Example 6

### Confirmation of Effect of Reducing Surface Free Energy in Mixed Solvent

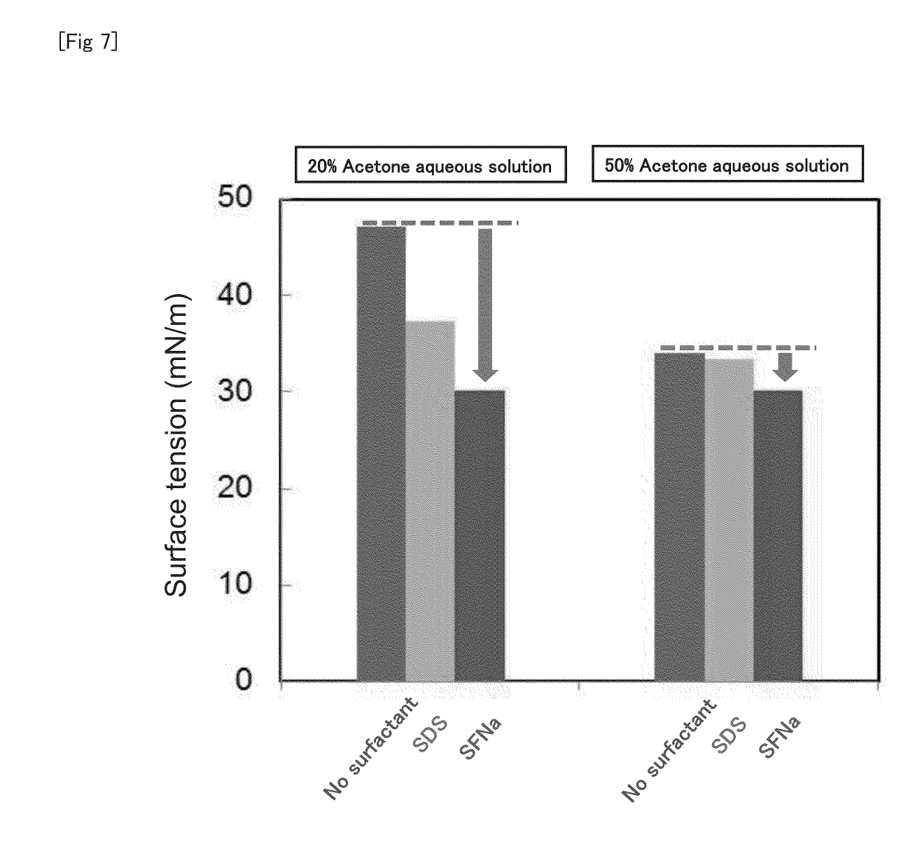

An experiment for confirmation of effect of reducing surface free energy by SFNa in a mixed solvent which contained acetone as a water-miscible solvent was also carried out, since the secondary structure formation was confirmed in the above-described Example 5. Specifically, SFNa and 20 vol % or 50 vol % acetone aqueous solution (10 mL) were added in a test tube so that SFNa concentration became 1 mass % similarly to the above-described Example 1. The mixture was stirred using a vortex mixer for 3 minutes. In addition, 1 mass % SDS solution was obtained as control by dissolving SDS in 20 vol % or 50 vol % acetone aqueous solution. The dispersion was transferred to a petri dish and left to stand similarly to the above-described Example 2. The surface tension was measured at 25° C. using a high function surface tension measuring device (“DY-500”, manufactured by Kyowa Interface Science Co., Ltd.). The result is shown in FIG. 7.

As FIG. 7, SDS reduced the surface tension of 20 vol % acetone aqueous solution; however, the effect of reducing surface free energy by SFNa was clearly superior to SDS. In addition, SDS could not reduce the surface tension of 50 vol % acetone aqueous solution. On the one hand, when SFNa was used, the surface tension could be clearly reduced. It was thus demonstrated that not only a micelle can be formed but also surface free energy can be remarkably reduced even in a mixed solvent of water and acetone by SFNa.

### Example 7

### Confirmation of Secondary Structure Formation in Organic Solvent

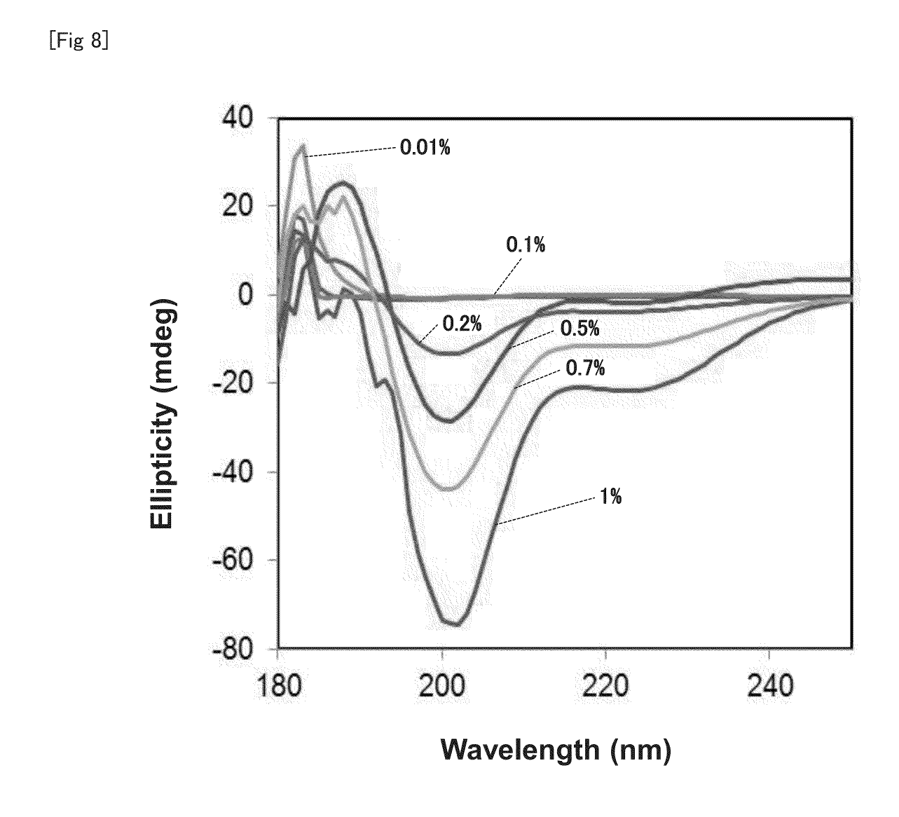

Similarly to the above-described Example 5, methanol (1 mL) and SFNa were measured off in a test tube, and the mixture was stirred using a vortex mixer for 3 minutes in order to prepare 0.01 to 1 mass % SFNa solutions in methanol. Then, secondary structure formation of SFNa in the prepared acetone solutions was measured using a circular dichroism dispersion meter (“J-820”, manufactured by JASCO Corporation). The result is shown in FIG. 8.

It was demonstrated in FIG. 8 that a negative peak was observed near 200 nm. It was clearly demonstrated from the result that SFNa also forms secondary structure in a methanol solution. In addition, it was confirmed that when the concentration of SFNa was higher, secondary structure formation is facilitated, since larger peak can be observed in such a case. It is experimentally demonstrated from the result that surface free energy of methanol can be reduced by the aggregate such as micelle of a stable secondary structure formed by SFNa even in methanol.

### Example 8

### Confirmation of Secondary Structure Formation in Organic Solvent

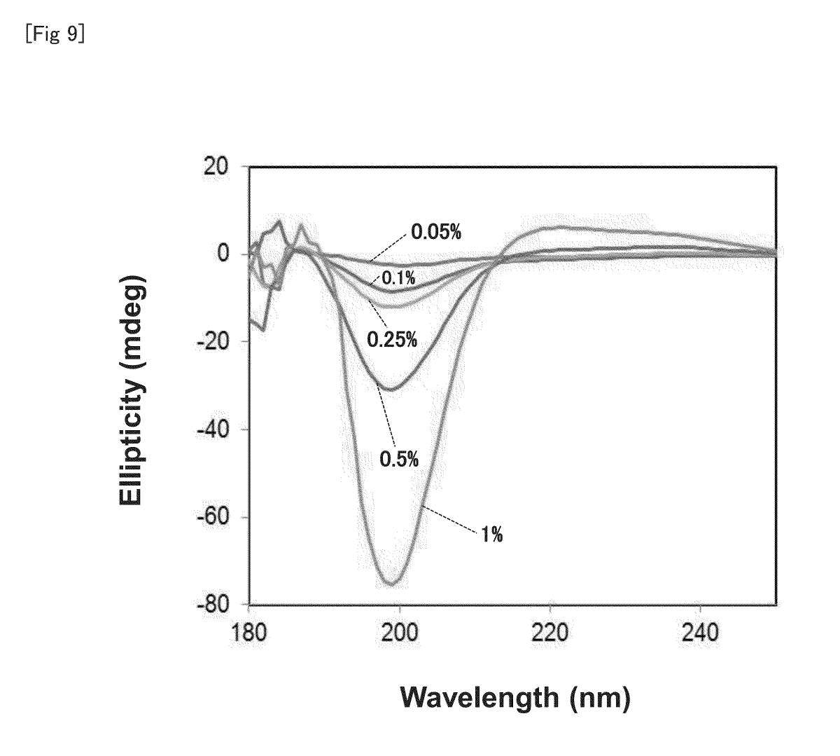

Similarly to the above-described Example 5, ethanol (1 mL) and SFNa were measured off in a test tube, and the mixture was stirred using a vortex mixer for 3 minutes in order to prepare 0.05 to 1 mass % SFNa solutions in ethanol. Then, secondary structure formation of SFNa in the prepared ethanol solutions was measured using a circular dichroism dispersion meter (“J-820”, manufactured by JASCO Corporation). The result is shown in FIG. 9.

It was demonstrated in FIG. 9 that a negative peak was observed near 200 nm. It was clearly demonstrated from the result that SFNa also forms secondary structure in ethanol solution. In addition, it was confirmed that when the concentration of SFNa was higher, secondary structure formation was facilitated, since larger peak could be observed. It is thus contemplated that surface free energy of ethanol can be reduced by the aggregate such as micelle of a stable secondary structure formed by SFNa even in ethanol.

### Example 9

### Confirmation of Secondary Structure Formation in Organic Solvent

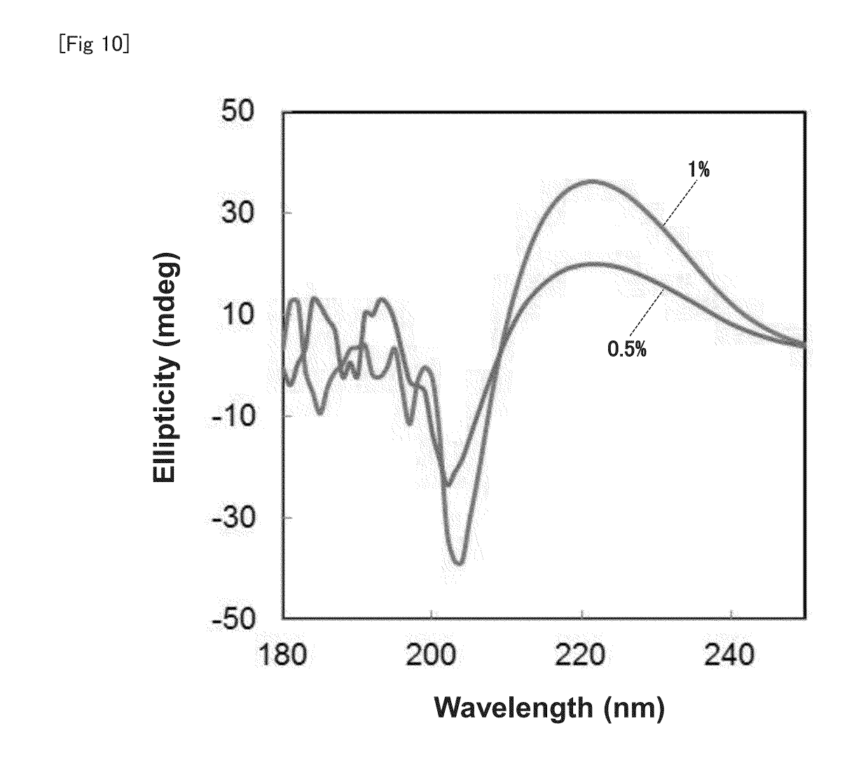

Similarly to the above-described Example 5, tetrahydrofuran (1 mL) and SFNa were measured off in a test tube, and the mixture was stirred using a vortex mixer for 3 minutes in order to prepare 0.5 mass % or 1 mass % SFNa solutions in tetrahydrofuran. Then, secondary structure formation of SFNa in the prepared tetrahydrofuran solutions was measured using a circular dichroism dispersion meter (“J-820”, manufactured by JASCO Corporation). The result is shown in FIG. 10.

It was demonstrated in FIG. 10 that a negative peak was observed near 200 nm. It was clearly demonstrated from the result that SFNa also forms secondary structure in tetrahydrofuran solution. In addition, it was confirmed that when the concentration of SFNa was higher, secondary structure formation was facilitated, since larger peak could be observed. It is thus contemplated that surface free energy of tetrahydrofuran can be reduced by the aggregate such as micelle of a stable secondary structure formed by SFNa even in tetrahydrofuran.

1. A method for reducing surface free energy of an organic solvent or a mixed solvent of water and a water-miscible solvent,
comprising the step of adding surfactin represented by the following formula (I) or a salt thereof to the organic solvent or mixed solvent, 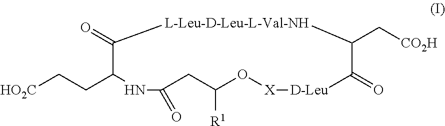
wherein ‘X’ is a residue of an amino acid selected from leucine, isoleucine and valine; and R¹ is a C₉₋₁₈ alkyl group.

2. The method according to claim 1, wherein the surfactin or salt thereof is added in an amount of not less than 0.01 mass % to the organic solvent or mixed solvent.

3. The method according to claim 1, wherein a ratio of the water-miscible solvent in the mixed solvent is adjusted to not less than 5 vol %.

4. A composition, comprising surfactin represented by the following formula (I) or a salt thereof and an organic solvent or a mixed solvent of water and a water-miscible solvent, 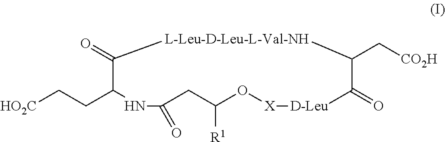
wherein ‘X’ is a residue of an amino acid selected from leucine, isoleucine and valine; and R¹ is a C₉₋₁₈ alkyl group.

5. The composition according to claim 4, wherein the surfactin or salt thereof is contained in an amount of not less than 0.01 mass % to the organic solvent or mixed solvent.

6. The composition according to claim 4, wherein a ratio of the water-miscible solvent in the mixed solvent is not less than 5 vol %.

7. A wet wiper, comprising the composition according to claim 4.

8. An antiseptic solution, comprising the composition according to claim 4.
